SEQUENCE LISTING (8 sequences)

SEQ ID NO: 1 - protein, 187 aa, Homo sapiens
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKLVKFISSELKESIESQFLELQREGEKQ

SEQ ID NO: 2 - protein, 179 aa, Mus musculus
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKERHAAEVRRNKELQVELSG

SEQ ID NO: 3 - DNA, 3509 bp, synthetic construct
  source 1..3509: mol_type = other DNA
tagctgcacaaacccagacctgggtgtggtcagccccacagggatagttaatccgaagcagcttcccgagcaggaggaagctcagagcacaaacctaggcctgggcgtggccactgggagctgctgatagcgaaccccagctcacaccagtttcttttttggtcgtgggaagaaaaacacatattatcctgttgtcacaagatctgtgaccttatatgaaaaaatgctagaattttttcattaaaaaagaaaatactgaactagccagtgacccagatgttttcagaacctagactggttctgtccattggaaaacctcggtgtctgcattaacttttcaccacactagagggcaatcatgttctctaaaaaagcagatgattgatgtaaacctagttccaaatattaactgtttaataaaatcttttcttttaccaggaacattcaagtgtttattcaataagctgatgccatgctttaccctagtggatgaacagagcttgtacaattttcaaggagacaggatgaaatgagtggtcataatctgaaagtagatacacgccctggttaattattccctgatggttttacttctcagttttattacattgttattataataccatttatgttacttctgagattttgtagtggataaatagtagaaaaatgtcagtagtaatagcaaagttatttagcagccgaatattttaatgcttaaaaataaaggaataaattaaagaaaatcattgtttacttcttcatcgattgaaatgtgccccctgttcagagcacatctgaatatcagagtctccacctgcagagaacatgcagcttagcgagtaaaacaggcaggtatgtgatactgaggaggtgtaccaaaaactgactgctgttatttttcccatcttctaagtctgtctttcttttccatttaaagatacctttttaaatctaatccaatgtgatttcaatctagttttatcagatttcaacaattattgagcatctccttgtagtggttttctgtttattagaaaatcgatgttaattttaacgaagtaagaagaaatatataagtataaactaattttgggtatcatcaaaagtggattttttaaatatgcattgatagaattattttttgattacattttatgtaattctaatccagctataaaatatttaatagtgtcatattactgtgttcctcaaactttgatgtgcatatgaattacctttgattttcattaaaatgcaaattctgattcaatacatctggcttgaggcagacattctgtcttccgaacaagctcccagatgatgctgattctgaccactaaacacatcagttttagggatattaacttgtaatatacaggtatccctcctggtaagctctggtattatgtcttaacatttttaaatctatggtaatctttacaaaatattttacttccgaactcatatacctggggattttattactctgggaattatgtgttctgccccatcactctctcttaattggatttttaaaattatattcatattgcaggactcggcagaagaccttcgagagaaaggtagaaaataagaatttggctctcacatgaggatcacccatgtcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttatatttattatttaccaaattattctaagagtatttcttcctgaataccatgtgagaaaattcttaagaatttattgagtatgactgtatatttgaaaagagtgttttcttctgcttatctaagccaataaaggatcttcattattcaattctaactttctaaggaagtcaacctacagatcagaaagaggatcttcaaggaatagcatcaaagacatagtcaggtctcccatgcagtgactggctgaccatgcagccattaccacctttctggaaatattatgctgcaaaaatgatacaatacacgaaatatctcaaattaaaaaatataacatttcccaaatagggcactaaaaacatgatcccaaataaaactagcttcagggtttgcagaatatactgttactcaacacaaagttggactaagtctcaaagttagccattcagttgttgttaacagttcatttcagggtctctcagaagctgggaaactttccatttttgcaatttcttgtacattgaaggaaaggaagacacacttaagacagcattacaaaagtaattcatgttttaaatgtttaattctggcagtcgggcagggctctctgtataacctcatttggagatgacaaaaatctaaacttgagggcctcgagccaataagtcttcctatttctttactcaaacattttcccgcaatggtgctttctttcaactgtttttctggtgtattcataaattccagattctctatgggaagtaacttttattgattgatttaacccttgtatagcacatataacatgcaaggcattgttctaagaactttccacatattaactgtgttaatcacttaataatcctaagtaggttctattacagatatggaaactgaggcacagaaagttgaagtatcttactcaaggtcacacagttagtcagatccagaatttgggcccaggccatctggcttcggaatccatctttcaccgattgctgctagtctcatatctgttccatgttagaggtgagctcccattgcagaggtcacacctgtgatatcaccattttatttaaacagaccagagatggtcttctcctttctgatcacagactcaccttgaagagaaaatacttccaaattgatgcctagttttaatagcttacctggggcttattcaaataattgccatgatttaggctttgggagaaagagagctatgaggccgtgtgggttgtaacgtatgagacacatggcgttctgcaggctcagcacagcatcgatttctggtgggaacacactctgatgaccagttccagaaataacattgacttaatctcctcagtcccatcatggttagcacatttcaaaatgcctccttaactacttccataggccagagatatttagttttaacattttgttgaataaaataaatttacacattcacatttaatataactattagatgttatttcaagattctcttcatattaccatcaaagcaggcaggcaggcaggagagaactgtaggaaggaccaggttttttgtttgtttgcttgtgtttgtggaaaaacacgcactgttgttacgatgtaatctgtaacattgtatgaatgtctgatccacagaaaacactcaaacccaaatgcttccagaaccgaagctatttgcctcctagttcttgcaattgggagaccatggagtctgcactaac

SEQ ID NO: 4 - DNA, 19 bp, Homo sapiens
  source 1..19: mol_type = genomic DNA
acatgaggatcacccatgt

SEQ ID NO: 5 - DNA, 24 bp, Mus musculus
  source 1..24: mol_type = genomic DNA
tgtgtgagcatgtgtgcgtgtgtg

SEQ ID NO: 6 - DNA, 222 bp, Homo sapiens
  source 1..222: mol_type = genomic DNA
gactcggcagaagaccttcgagagaaaggtagaaaataagaatttggctctcacatgaggatcacccatgtcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctc

SEQ ID NO: 7 - DNA, 20 bp, synthetic construct
  source 1..20: mol_type = other DNA
ccctcctggtaagctctggt

SEQ ID NO: 8 - DNA, 24 bp, synthetic construct
  source 1..24: mol_type = other DNA
ggaagtcaacctacagatcagaaa

(19) United States
(12) Patent Application Publication
(10) Pub. No.: US 2025/0040523 A1
(43) Pub. Date: Feb. 6, 2025

(54) METHODS AND COMPOSITIONS RELATING TO HUMANIZED STATHMIN2 MOUSE MODEL WITH DISRUPTED TDP-43 BINDING SITES

(71) Applicant: The Jackson Laboratory, Bar Harbor, ME (US)

(72) Inventor: Cathleen Marie Lutz, Bar Harbor, ME (US)

(73) Assignee: The Jackson Laboratory, Bar Harbor, ME (US)

(21) Appl. No.: 18/711,686

(22) PCT Filed: Nov. 21, 2022
(86) PCT No.: PCT/US2022/080224

§ 371 Date: May 20, 2024

Related U.S. Application Data
(60) Provisional application No. 63/282,023, filed on Nov. 22, 2021.

(51) Int. Cl. A01K 67/0278; C07K 14/47

(52) U.S. Cl. CPC A01K 67/0278; C07K 14/47; A01K 2207/15; A01K 2217/072; A01K 2227/105; A01K 2267/03

(57) ABSTRACT

The present disclosure provides mouse models comprising an endogenous Stathmin2 (Stmn2) gene that comprises a human exon 2a polynucleotide sequence.

### RELATED APPLICATION

This application claims the benefit under 35 U.S.C. § 119(e) of U.S. provisional application No. 63/282,023, filed Nov. 22, 2021, which is incorporated by reference herein in its entirety.

### GOVERNMENT LICENSE RIGHTS

This invention was made with government support under Grant No. R01 NS124203 awarded by National Institutes of Health. The government has certain rights in the invention.

### REFERENCE TO AN ELECTRONIC SEQUENCE LISTING

The contents of the electronic sequence listing (J022770109WO00-SEQ-HJD.xml; Size: 12,142 bytes; and Date of Creation: Nov. 21, 2022) is herein incorporated by reference in its entirety.

### BACKGROUND

Neurodegenerative disorders, such as amyotrophic lateral sclerosis and frontotemporal dementia, affect millions of people worldwide. Neurodegenerative diseases occur when nerve cells in the brain or peripheral nervous system lose function over time and ultimately die. Over the next 40 years, the prevalence of neurodegenerative diseases is estimated to double in the US. Although treatments may help relieve some of the physical or mental symptoms associated with neurodegenerative diseases, there is currently no way to slow disease progression and no known cures. There is a continuing need for non-human animal models that recapitulate predominant disease mechanisms observed in neurodegenerative disease patients in order to develop therapeutic approaches.

### SUMMARY

The present disclosure provides, in some aspects, mouse models of Tar DNA-binding protein 43 (TDP-43) proteinopathies, such as amyotrophic lateral sclerosis and frontotemporal dementia. These mouse models are based in part on experimental evidence showing Stathmin-2 (Stmn2) gene disruption in such proteinopathies. This gene undergoes cryptic splicing of its mRNA, resulting in a truncated mRNA. The mouse models described herein recapitulate a disease mechanism central to a broad spectrum of neurodegenerative conditions and may be used, for example, to develop therapies in TDP-43 proteinopathies through identification of agents (e.g., antisense oligonucleotides and/or small molecule drugs) that block cryptic (aberrant) splicing of Stmn2 pre-mRNA.

Some aspects of the present disclosure provide a mouse model of Tar DNA-binding protein 43 (TDP-43) proteinopathies comprising a humanized stathmin-2 (Stmn2) gene comprising an exogenous polynucleotide sequence, wherein the exogenous polynucleotide sequence comprises human STMN2 exon 2a sequence.

In some embodiments, the human STMN2 exon 2a sequence has a length of about 200 to about 300 nucleotides, optionally a length of about 220 to about 225, preferably 222 nucleotides.

In some embodiments, the exogenous polynucleotide sequence comprises human genomic DNA flanking the human STMN2 exon 2a sequence.

In some embodiments, the human STMN2 exon 2a sequence comprises a human MS2 stem loop sequence.

In some embodiments, the human MS2 stem loop sequence comprises the sequence of 5′-ACATGAGGATCACCCATGT-3′ (SEQ ID NO: 4).

In some embodiments, the human MS2 stem loop sequence replaces TDP-43 binding sequence.

In some embodiments, the TDP-43 binding sequence comprises the sequence of 5′-TGTGTGAGCATGTGTGCGTGTGTG-3′ (SEQ ID NO: 5).

In some embodiments, the human STMN2 exon 2a sequence is in intron 1 of the mouse Stmn2 gene.

In some embodiments, the mouse model has a C57BL/6 genetic background.

In some embodiments, the genotype of the mouse is C57BL/6J-Stmn2^{em8(STMN2} [symbol] ^{)Lutzy}/Mmjax.

In some embodiments, the TDP-43 proteinopathies are selected from amyotrophic lateral sclerosis (ALS) and frontotemporal degeneration (FTD).

Other aspects provide a method comprising administering a candidate therapeutic agent to the mouse of any one of the preceding claims, and assaying the mouse for a modified phenotype.

In some embodiments, the candidate therapeutic agent blocks aberrant/cryptic splicing of Stmn2 pre-mRNA.

In some embodiments, the candidate therapeutic is an antisense oligonucleotide (ASO) that binds to the human exon2a polynucleotide sequence.

In some embodiments, the candidate therapeutic is a small molecule drug.

In some embodiments, the phenotype is selected from a behavioral phenotype, a pathological phenotype, a cognitive deficit, a motor deficit, motor neuron degeneration, neuromuscular denervation.

In some embodiments, the phenotype is a motor deficit.

In some embodiments, the motor deficit is selected from tremors, paralysis, abnormal gait, or hindlimb clasping.

In some embodiments, the motor deficit is assayed by open field, grip strength, or rotarod analyses.

In some embodiments, the phenotype is a cognitive deficit.

Yet other aspects provide a method of producing the mouse of any one of claims 1-11, the method comprising introducing the exogenous polynucleotide sequence into an intron of the endogenous Stmn2 gene of the mouse, optionally using a gene editing technique.

### BRIEF DESCRIPTION OF THE DRAWINGS

FIG. 1A shows a schematic of the human STMN2 gene. FIG. 1B shows a schematic of the mouse Stmn2 gene.

FIG. 2A shows a schematic of a mouse Stmn2 allele that includes a 394 base pair segment of intron 1 of the human STMN2. This modified allele is referred to herein as a “humanized mouse Stmn2 allele.” FIG. 2B shows a schematic of the humanized mouse Stmn2 allele in which the human TDP-43 binding sites have been replaced by MS2 binding sites, leading to constitutive inclusion of exon 2 (from top to bottom, SEQ ID NOs: 5 and 4).

FIGS. 3A-3D show a schematic and data relating to the generation and in vitro validation of humanized Stmn2 mice (i.e., mice that include the humanized mouse Stmn2 allele). Primary cortical neurons were generated from mouse embryos edited to carry the human exon 2a genomic region within intron 1 of the stathmin-2 mouse gene (Stmn2^{Hum/+} mice) and treated during 12 days with 1 or 3 μM of an RNase-H dependent ASO targeting mouse TDP-43 (FIG. 3A). Quantitative RT-PCR showing efficient reduction of TDP-43 (FIG. 3B) leading to reduced levels of the full length stathmin-2 mRNA (n=3 biological replicates per condition (FIG. 3C); [symbol] p<0.05, [symbol] [symbol] [symbol] p<0.001, one way ANOVA). Reduction of stathmin-2 is mediated by abnormal splicing between murine exon 1 and human exon 2a upon TDP-43 loss detected by RT-PCR in neurons from heterozygous Stmn2^{Hum/+} but not from wild-type embryos (FIG. 3D).

FIGS. 4A-4C show data from mouse neuroblastoma cells edited to include human exon 2a with TDP-43 binding sites replaced by MS2 sites, demonstrating constitutional mis-splicing and reduction of Stmn2. Quantitative RT-PCR for full length mouse Stmn2 in N2A neuroblastoma clones after CRISPR/Cas9 editing to introduce human exon 2a without TDP-43 binding sites (see FIG. 2B) in one allele (Het) or both alleles (Homo) compared to wild-type (WT) isogenic clones (FIG. 4A) ( [symbol] [symbol] p<0.001, one way ANOVA). RT-PCR showing constitutive splicing between murine exon 1 and human exon 2a in heterozygous and homozygous clones (FIG. 4B). Constitutive misplicing and reduced levels of stathmin-2 mRNA leads to 50% reduction or complete loss of STMN2 protein, respectively, in heterozygous and homozygous cells (FIG. 4C).

### DETAILED DESCRIPTION

Amyotrophic lateral sclerosis (ALS), a disease in which premature loss of upper and lower motor neurons leads to fatal paralysis, is now recognized to have clinical, genetic and pathological overlap with Frontotemporal degeneration (FTD), a neurodegenerative disorder characterized by behavioral and language dysfunction. What underlies age-dependent dysfunction of motor or cortical neurons remains unsolved. A landmark contribution to understanding cellular mechanisms of ALS and FTD came from the discovery of cytoplasmic accumulation and nuclear loss of the RNA binding protein TDP-43 from affected neurons in most instances of ALS (>95%), as well as in approximately 50% of patients with FTD. TDP-43 disruption represents a common pathological hallmark in both sporadic and familial forms of ALS and FTD including patients with mutations in the genes encoding for TDP-43 (TARDBP) and progranulin (GRN) and patients with G4C2 repeat expansions in the C9ORF72 gene. TDP-43 pathology is reported in a large spectrum of neurodegenerative conditions, referred as TDP-43 proteinopathies, that include ALS, FTD, and various forms of parkinsonism and dementia. More than 30% of patients have Alzheimer's disease or limbic-predominant age-related TDP-43 encephalopathy.

Widely expressed and predominantly nuclear, TDP-43 is an RNA binding protein playing a role in different aspects of RNA metabolism regulation. In patients, the protein is detectable in the nuclei of unaffected neurons but is cleared from nuclei in neurons containing cytoplasmic aggregations, evidence strongly supporting that TDP-43 loss of function is a key aspect of disease mechanism underlying ALS and FTD pathogenesis. Genome-wide approaches have been used to define binding sites of TDP-43 on thousands of RNA targets, with GU-rich sequences as preferred binding sites, and to define the influence of TDP-43 disruption on RNA splicing and expression of mouse and human transcripts.

TDP-43 is an essential protein, and its ubiquitous deletion in mice is embryonically lethal. Numerous transgenic rodents expressing human TDP-43 have been generated, with or without disease-causing mutations and under various promoters. Overexpression of TDP-43 induces a severe lethal phenotype independent of the presence of mutation. However, mice expressing levels close to endogenous TDP-43 develop mutant and age-dependent neurological phenotypes, including mild motor and cognitive deficits, motor neuron degeneration, and neuromuscular denervation, but without paralysis or reduced lifespan.

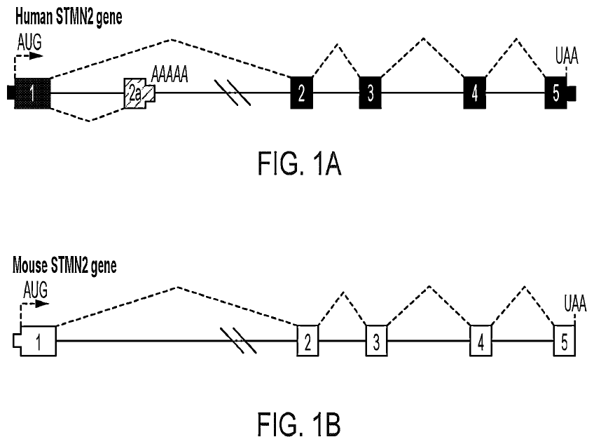

An important caveat for animal modeling of TDP-43 proteinopathies is that the repertoire of RNAs bound by TDP-43 differs between species with RNA processing alterations elicited by TDP-43 dysfunction distinct between mice and humans. Indeed, recent data shows that the human RNA most affected by TDP-43 disruption encodes the neuronal growth-associated factor stathmin-2 (also known as SCG10), but stathmin-2 RNAs are neither bound nor regulated by TDP-43 in rodents. Abnormal processing of stathmin-2 is not recapitulated in mice expressing TDP-43 transgenes or in TDP-43 deficient mice, as the cryptic polyadenylation signal and three GU-rich TDP-43 binding sites in intron 1 of the human stathmin-2 gene are not found in the corresponding mouse intron (FIGS. 1A and 1B). Consistently, stathmin-2 pre-mRNA is not bound by TDP-43 in mice, and stathmin-2 mRNA level is not altered after siRNA or ASO-mediated reduction of TDP-43 in mouse N2A cells and in the central nervous system of wild-type mice. See Melamed et al. (2019) Nat. Neurosci., incorporated herein by reference.

### I. Tar DNA-Binding Protein 43 (TDP-43) and Stathmin-2

A common neuropathological feature of several neurodegenerative diseases is the mislocalization and aggregation of Tar DNA-binding protein 43 (TDP-43), which is a member of the family of heterogenous nuclear ribonucleoproteins (hnRNPs) and is normally ubiquitously expressed in nuclei throughout the CNS. Intensive investigations regarding the normal function of TDP-43 and its role in the pathogenesis of neurodegeneration have identified a variety of pathways in which TDP-43 perturbations lead to neuronal toxicity. Originally identified as the major component of ubiquitinated cytoplasmic inclusions in amyotrophic lateral sclerosis (ALS) and frontotemporal lobar degeneration (FTD), TDP-43 pathology is now recognized as a neuropathological feature of a substantial proportion of cases of Alzheimer's disease as well as Parkinson's and Huntington's diseases, and even the degenerative muscle disease inclusion body myopathy (see Glass J D J Clin Invest. 2020 Nov. 2; 130(11):5677-5680).

TDP-43 is a transcription suppressor. In the absence of TDP-43, i.e., when TDP-43 is cleared from the nucleus as it mislocalizes to the cytoplasm, cryptic exons are exposed and transcribed, creating aberrant mRNAs that lead to neurotoxicity in ALS models. Recently, this TDP-43 suppressor function was specifically implicated in suppressing a cryptic early polyadenylation site in the pre-mRNA for the protein stathmin-2 (STMN2). Loss of TDP-43 allows incorporation of a premature poly A tail, resulting in a truncated and nonfunctional form of STMN2 (Klim J R et al., Nat Neurosci. 2019; 22(2):167-179 and Melamed Z, et al. Nat Neurosci. 2019; 22(2):180-190). STMN2 is a highly conserved cytosolic protein essential for axonal outgrowth and maintenance. STMN2-knockout mice develop late-onset, predominantly motor axonopathy (Liedtke W et al. Am J Pathol. 2002; 160(2):469-480) paralleling that seen in human ALS.

Among the four members of the stathmin genes family, each encoded from a different genomic locus, stathmin-2 is the most prominently expressed in both mouse and human motor neurons. Indeed, stathmin-2 is among the top 25 most enriched mRNAs in this neuronal population that is specifically affected in ALS. Stathmin-2 has been proposed to play an important role in neurite outgrowth48, most likely by promoting microtubule dynamics in axonal growth cones. It binds α/β-tubulin dimers and was shown to be essential for axonal regeneration. Upon axonal injury, stathmin-2 is upregulated and recruited to growth cones of regenerating axons, and it was determined that it accumulates at motor axon termini of neuromuscular junctions (NMJs) in adult mice.

Below is an exemplary human Stathmin-2 protein sequence:

(SEQ ID NO: 1)
MAKTAMAYKEKMKELSMLSLICSCFYPEPRNINIYTYDDMEVKQI
NKRASGQAFELILKPPSPISEAPRTLASPKKKDLSLEEIQKKLEA
AEERRKSQEAQVLKQLAEKREHEREVLQKALEENNNFSKMAEEKL
ILKMEQIKENREANLAAIIERLQEKLVKFISSELKESIESQFLEL
QREGEKQ

Below is an exemplary mouse Stathmin-2 protein sequence:

(SEQ ID NO: 2)
MAKTAMAYKEKMKELSMLSLICSCFYPEPRNINIYTYDDMEVKQI
NKRASGQAFELILKPPSPISEAPRTLASPKKKDLSLEEIQKKLEA
AEERRKSQEAQVLKQLAEKREHEREVLQKALEENNNFSKMAEEKL
ILKMEQIKENREANLAAIIERLQEKERHAAEVRRNKELQVELSG

In some embodiments, the mouse (mouse model) provided herein has the genotype C57BL/6J-Stmn2^{em8(STMN2} [symbol] ^{)Lutzy}/Mmjax (common name Stmn2^{em8(STMN} [symbol] ⁾, e.g., MMRRC Strain #069792-JAX). Stmn2^{em8(STMN2} [symbol] ⁾ is a CRISPR/cas9 generated mutant of the stathmin-like 2 (Stmn2) gene carrying 222 nucleotides of human STMN2 exon 2a. The STMN2 sequence is modified to include the human MS2 stem loop sequence and replace the TDP-43 binding element. These mice may be useful in pre-clinical studies of Amyotrophic Lateral Sclerosis (ALS).

The Stmn2^{em8(STMN2} [symbol] ⁾ allele was generated using CRISPR/cas9 endonuclease-mediated genome editing. Guide RNAs were selected to target intron 1 of the stathmin-like 2 gene (Stmn2) and insert a modified human SMNT2 exon 2a. The humanized/mutant exon 2a in this strain is 222nt in length and is flanked by 1491 bp and 1548 bp of human genomic DNA, located 5′- and 3′- of exon 2a, respectively. The 19-nt MS2 stem loop contains the sequence 5′-ACATGAGGATCACCCATGT-3′ (SEQ ID NO: 4) was used to replace the 24-nt TDP-43 binding sequence 5′-TGTGTGAGCATGTGTGCGTGTGTG-3′ (SEQ ID NO: 5) in the 3′-UTR of the exon2a-containing mouse-human hybrid STMN2 prematurely terminated mRNA transcript. The donor plasmid, both guides and cas9 nuclease were introduced into single cell C57BL/6J zygotes and transferred to pseudopregnant females. Note that the flanking mouse intron 1 sequences present in the donor plasmid was derived from N2A cell line. There are three SNPs in the left arm that differ between the C57BL/6J sequence reported in Ensembl. It is not expected that any of these SNPs impact the usage of exon 2a. Progeny were screened by DNA sequencing to identify correctly targeted pups, which were then bred to C57BL/6J mice for germline transmission. N1 progeny derived from a single correctly targeted founder carrying the allele were backcrossed to C57BL/6J for at least two generations to establish the colony.

Predicted Genomic Sequence of the Humanized exon2A (Mutant TDP-43) in Mouse:

(SEQ ID NO: 3)
TAGCTGCACAAACCCAGACCTGGGTGTGGTCAGCCCCACAGGGAT
AGTTAATCCGAAGCAGCTTCCCGAGCAGGAGGAAGCTCAGAGCAC
AAACCTAGGCCTGGGCGTGGCCACTGGGAGCTGCTGATAGCGAAC
CCCAGCTCACACCAGTTTCTTTTTTGGTCGTGGGAAGAAAAACAC
ATATTATCCTGTTGTCACAAGATCTGTGACCTTATATGAAAAAAT
GCTAGAATTTTTTCATTAAAAAAGAAAATACTGAACTAGCCAGTG
ACCCAGATGTTTTCAGAACCTAGACTGGTTCTGTCCATTGGAAAA
CCTCGGTGTCTGCATTAACTTTTCACCACACTAGAGGGCAATCAT
GTTCTCTAAAAAAGCAGATGATTGATGTAAACCTAGTTCCAAATA
TTAACTGTTTAATAAAATCTTTTCTTTTACCAGGAACATTCAAGT
GTTTATTCAATAAGCTGATGCCATGCTTTACCCTAGTGGATGAAC
AGAGCTTGTACAATTTTCAAGGAGACAGGATGAAATGAGTGGTCA
TAATCTGAAAGTAGATACACGCCCTGGTTAATTATTCCCTGATGG
TTTTACTTCTCAGTTTTATTACATTGTTATTATAATACCATTTAT
GTTACTTCTGAGATTTTGTAGTGGATAAATAGTAGAAAAATGTCA
GTAGTAATAGCAAAGTTATTTAGCAGCCGAATATTTTAATGCTTA
AAAATAAAGGAATAAATTAAAGAAAATCATTGTTTACTTCTTCAT
CGATTGAAATGTGCCCCCTGTTCAGAGCACATCTGAATATCAGAG
TCTCCACCTGCAGAGAACATGCAGCTTAGCGAGTAAAACAGGCAG
GTATGTGATACTGAGGAGGTGTACCAAAAACTGACTGCTGTTATT
TTTCCCATCTTCTAAGTCTGTCTTTCTTTTCCATTTAAAGATACC
TTTTTAAATCTAATCCAATGTGATTTCAATCTAGTTTTATCAGAT
TTCAACAATTATTGAGCATCTCCTTGTAGTGGTTTTCTGTTTATT
AGAAAATCGATGTTAATTTTAACGAAGTAAGAAGAAATATATAAG
TATAAACTAATTTTGGGTATCATCAAAAGTGGATTTTTTAAATAT
GCATTGATAGAATTATTTTTTGATTACATTTTATGTAATTCTAAT
CCAGCTATAAAATATTTAATAGTGTCATATTACTGTGTTCCTCAA
ACTTTGATGTGCATATGAATTACCTTTGATTTTCATTAAAATGCA
AATTCTGATTCAATACATCTGGCTTGAGGCAGACATTCTGTCTTC
CGAACAAGCTCCCAGATGATGCTGATTCTGACCACTAAACACATC
AGTTTTAGGGATATTAACTTGTAATATACAGGTATCCCTCCTGGT
AAGCTCTGGTATTATGTCTTAACATTTTTAAATCTATGGTAATCT
TTACAAAATATTTTACTTCCGAACTCATATACCTGGGGATTTTAT
TACTCTGGGAATTATGTGTTCTGCCCCATCACTCTCTCTTAATTG
GATTTTTAAAATTATATTCATATTGCAGGACTCGGCAGAAGACCT
TCGAGAGAAAGGTAGAAAATAAGAATTTGGCTCTCACATGAGGAT
CACCCATGTCGAGAGAGAGAGACAGACAGCCTGCCTAAGAAGAAA
TGAATGTGAATGCGGCTTGTGGCACAGTTGACAAGGATGATAAAT
CAATAATGCAAGCTTACTATCATTTATGAATAGCAATACTGAAGA
AATTAAAACAAAAGATTGCTGTCTCAATATATCTTATATTTATTA
TTTACCAAATTATTCTAAGAGTATTTCTTCCTGAATACCATGTGA
GAAAATTCTTAAGAATTTATTGAGTATGACTGTATATTTGAAAAG
AGTGTTTTCTTCTGCTTATCTAAGCCAATAAAGGATCTTCATTAT
TCAATTCTAACTTTCTAAGGAAGTCAACCTACAGATCAGAAAGAG
GATCTTCAAGGAATAGCATCAAAGACATAGTCAGGTCTCCCATGC
AGTGACTGGCTGACCATGCAGCCATTACCACCTTTCTGGAAATAT
TATGCTGCAAAAATGATACAATACACGAAATATCTCAAATTAAAA
AATATAACATTTCCCAAATAGGGCACTAAAAACATGATCCCAAAT
AAAACTAGCTTCAGGGTTTGCAGAATATACTGTTACTCAACACAA
AGTTGGACTAAGTCTCAAAGTTAGCCATTCAGTTGTTGTTAACAG
TTCATTTCAGGGTCTCTCAGAAGCTGGGAAACTTTCCATTTTTGC
AATTTCTTGTACATTGAAGGAAAGGAAGACACACTTAAGACAGCA
TTACAAAAGTAATTCATGTTTTAAATGTTTAATTCTGGCAGTCGG
GCAGGGCTCTCTGTATAACCTCATTTGGAGATGACAAAAATCTAA
ACTTGAGGGCCTCGAGCCAATAAGTCTTCCTATTTCTTTACTCAA
ACATTTTCCCGCAATGGTGCTTTCTTTCAACTGTTTTTCTGGTGT
ATTCATAAATTCCAGATTCTCTATGGGAAGTAACTTTTATTGATT
GATTTAACCCTTGTATAGCACATATAACATGCAAGGCATTGTTCT
AAGAACTTTCCACATATTAACTGTGTTAATCACTTAATAATCCTA
AGTAGGTTCTATTACAGATATGGAAACTGAGGCACAGAAAGTTGA
AGTATCTTACTCAAGGTCACACAGTTAGTCAGATCCAGAATTTGG
GCCCAGGCCATCTGGCTTCGGAATCCATCTTTCACCGATTGCTGC
TAGTCTCATATCTGTTCCATGTTAGAGGTGAGCTCCCATTGCAGA
GGTCACACCTGTGATATCACCATTTTATTTAAACAGACCAGAGAT
GGTCTTCTCCTTTCTGATCACAGACTCACCTTGAAGAGAAAATAC
TTCCAAATTGATGCCTAGTTTTAATAGCTTACCTGGGGCTTATTC
AAATAATTGCCATGATTTAGGCTTTGGGAGAAAGAGAGCTATGAG
GCCGTGTGGGTTGTAACGTATGAGACACATGGCGTTCTGCAGGCT
CAGCACAGCATCGATTTCTGGTGGGAACACACTCTGATGACCAGT
TCCAGAAATAACATTGACTTAATCTCCTCAGTCCCATCATGGTTA
GCACATTTCAAAATGCCTCCTTAACTACTTCCATAGGCCAGAGAT
ATTTAGTTTTAACATTTTGTTGAATAAAATAAATTTACACATTCA
CATTTAATATAACTATTAGATGTTATTTCAAGATTCTCTTCATAT
TACCATCAAAGCAGGCAGGCAGGCAGGAGAGAACTGTAGGAAGGA
CCAGGTTTTTTGTTTGTTTGCTTGTGTTTGTGGAAAAACACGCAC
TGTTGTTACGATGTAATCTGTAACATTGTATGAATGTCTGATCCA
CAGAAAACACTCAAACCCAAATGCTTCCAGAACCGAAGCTATTTG
CCTCCTAGTTCTTGCAATTGGGAGACCATGGAGTCTGCACTAAC

The inserted MS2 binding sequence, ACATGAGGATCACCCATGT (SEQ ID NO: 4), disrupts TDP-43 binding element by replacement of the sequence

(SEQ ID NO: 5)
TGTGTGAGCATGTGTGCGTGTGTG.

The human Exon 2A sequence includes the following sequence:

(SEQ ID NO: 6)
GACTCGGCAGAAGACCTTCGAGAGAAAGGTAGAAAATAAGAATTT
GGCTCTCACATGAGGATCACCCATGTCGAGAGAGAGAGACAGACA
GCCTGCCTAAGAAGAAATGAATGTGAATGCGGCTTGTGGCACAGT
TGACAAGGATGATAAATCAATAATGCAAGCTTACTATCATTTATG
AATAGCAATACTGAAGAAATTAAAACAAAAGATTGCTGTCTC.

The polyA signal sequence is ATTAAA.

The Mut primer binding sites are CCCTCCTGGTAAGCTCTGGT (SEQ ID NO: 7) and GGAAGTCAACCTACAGATCAGAAA (SEQ ID NO: 8).

The flanking mouse intron sequences are underlined.

Two independent strategies have been used to identify the mRNA encoding stathmin-2 to be the most affected human RNA when TDP-43 is reduced: (1) depletion of TDP-43 by siRNA in a human neuronal cell line and (2) genome editing to introduce an ALS-causing mutation TDP-43N352S into both endogenous TDP-43 gene loci. Using RNAseq, stathmin-2 was the mRNA most affected by reduction in TDP-43, with a corresponding almost complete loss of the 22 kD stathmin-2 protein (Melamed Z, et al. Nat Neurosci. 2019; 22(2):180-190). The stathmin-2 gene contains 5 annotated exons. Reduction or mutation in TDP-43, however, induced a new spliced exon, with RNA-seq reads mapping within intron one (Melamed Z, et al. 2019). This new exon, herein called “exon 2a”, was absent in wild type cells, but appeared either when TDP-43 was depleted or when endogenous TDP-43 was edited to carry the N352S mutation. Use of a cryptic 3′ splice site produced RNAs with exon 1 ligated to exon 2a in SH-SY5Y cells after TDP-43 depletion or in the presence of the TDP-43N352S mutation (confirmed by RT-), but no RNAs containing exon 2a ligated to any downstream exon 2 were identified. Rather, it was determined that the 3′ boundary of exon 2a is produced by usage of a cryptic polyadenylation site within what is normally intron 1 (Melamed Z, et al. 2019). Three “GUGUGU” hexamers marking potential binding sites of TDP-43 were identified in a region between the cryptic 3′ splice site and premature polyA site whose use produces exon 2a. Indeed, an analysis of ultraviolet cross-linking and immuno-precipitation (iCLIP) data for TDP-43 in human SH-SY5Y cells confirmed (FIG. 1e) that TDP-43 does bind to the stathmin-2 pre-mRNA at these three GUGUGU sequences (the only sites of TDP-43 binding to the stathmin-2 pre-mRNA), thus confirming physical interaction of the pre-mRNA with TDP-43. Altogether, reduction or disease-linked mutation in TDP-43 drives aberrant splicing and premature polyadenylation of human stathmin-2 pre-mRNA, thereby silencing stathmin-2 production (Melamed Z, et al. 2019).

### II. Mouse Models

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson M T, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

The mouse models provide herein are transgenic mouse models that express an endogenous Stathmin-2 gene that has been genetically modified to include an exogenous human Stathmin-2 polynucleotide sequence, referred to herein as a ‘humanized Stathmin-2 gene’. A transgenic mouse is a mouse having an exogenous nucleic acid (e.g., transgene or region of a transgene) in (integrated into) its genome.

Methods of producing transgenic mice are well-known. Three conventional methods used for the production of transgenic mice include DNA microinjection (Gordon and Ruddle, Science 1981: 214: 1244-124, incorporated herein by reference), embryonic stem cell-mediated gene transfer (Gossler et al., Proc. Natl. Acad. Sci. 1986, 83: 9065-9069, incorporated herein by reference) and retrovirus-mediated gene transfer (Jaenisch, Proc. Natl. Acad. Sci. 1976, 73: 1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a nucleic acid encoding humanized Stathmin-2 gene may be confirmed, for example, using any number of genotyping methods (e.g., sequencing and/or genomic PCR).

### III. Nucleic Acids: Engineering and Delivery

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome (e.g., in a coding region, non-coding region, and/or regulatory region), relative to the corresponding wild-type nucleic acid (e.g., the naturally-occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A nucleic acid, such as an allele or alleles of a gene, may be modified such that it does not produce a detectable level of a functional gene product (e.g., a functional protein). Thus, an inactivated allele is an allele that does not produce a detectable level of a functional gene product (e.g., a functional protein). A detectable level of a protein is any level of protein detected using a standard protein detection assay, such as flow cytometry and/or an ELISA. In some embodiments, an inactivated allele is not transcribed. In some embodiments, an inactivated allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example. Methods for delivering nucleic acids to mouse embryos for the production of transgenic mice include, but are not limited to, electroporation (see, e.g., Wang W et al. J Genet Genomics 2016; 43(5):319-27; WO 2016/054032; and WO 2017/124086, each of which is incorporated herein by reference), DNA microinjection (see, e.g., Gordon and Ruddle, Science 1981: 214: 1244-124, incorporated herein by reference), embryonic stem cell-mediated gene transfer (see, e.g., Gossler et al., Proc. Natl. Acad. Sci. 1986; 83: 9065-9069, incorporated herein by reference), and retrovirus-mediated gene transfer (see, e.g., Jaenisch, Proc. Natl. Acad. Sci. 1976; 73: 1260-1264, incorporated herein by reference), any of which may be used as provided herein

### IV. Genomic Editing Methods

Engineered nucleic acids, such as guide RNAs, donor polynucleotides, and other nucleic acid coding sequences, for example, may be introduced to a genome of an embryo using any suitable method. The present application contemplates the use of a variety of gene editing technologies, for example, to introduce nucleic acids into the genome of an embryo to produce a transgenic rodent. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405, each of which is incorporated by reference herein.

In some embodiments, a CRISPR system is used to edit the genome of mouse embryos provided herein. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein, one or multiple guide RNAs (gRNAs), and/or a donor nucleic acid can be delivered, e.g., injected or electroporated, directly into mouse embryos at the one-cell (zygote) stage or a later stage to facilitate homology directed repair (HDR), for example, to introduce an engineered nucleic acid (e.g., donor nucleic acid) into the genome.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337: 816-821 and Deltcheva et al., Nature, 2100; 471: 602-607, each of which is incorporated by reference herein.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

A donor nucleic acid typically includes a sequence of interest flanked by homology arms. Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

### V. Methods of Use

The mouse models provided herein may be used, in some embodiments, to determine how stathmin-2 contributes to neuronal function. In other embodiments, the mouse models may be used to develop therapeutic approaches for treating neurodegenerative diseases, such as TDP-43 proteinopathies that include, for example, ALS, FTD, and various forms of parkinsonism. Thus, provided herein, in some aspects, are methods comprising administering to a mouse model a candidate therapeutic molecule, and assaying for a modified phenotype in the mouse.

Among the most promising translational strategies of gene therapy for neurodegenerative diseases, antisense oligonucleotides (ASOs) have emerged as a viable, life extending therapeutic approach by correcting fatal gene expression or RNA processing defects. ASOs are short single-stranded chemically modified nucleic acids that bind to their complement target RNA molecule, create a chimeric duplex that for unmodified ASOs are recognized by the cellular endonuclease RNase H as a DNA-RNA hybrid, leading to cleavage and degradation of the target RNA, but not the ASO62. In a pioneering effort of ASOs administration in the central nervous systems, now in clinical trials for the treatment of familial ALS patients with mutation in the superoxide dismutase 1 (SOD1) gene, lowering the mRNA encoding SOD1 resulted in a significant slowing of disease progression. ASOs have since been utilized for targeting genetic causes of several neurological diseases including Huntington's disease, C9ORF72-ALS/FTD67-70, Taupathies, sporadic ALS and spinocerebellar ataxia, Batten's disease, and Pelizaeus-Merzbacher disease. When modified so as not to be recognized by RNase H, ASOs can modulate pre-mRNA processing. Indeed, the first treatment for the fatal neurodevelopmental disease spinal muscular atrophy (SMA) uses this strategy. The SMA drug Spinraza (also known as Nusinersen) is a chemically modified ASO-based drug that binds its targeted mRNA without recruiting the RNase H enzyme, leading to splicing modulation but not mRNA degradation.

Reduced levels in stathmin-2 are a hallmark of ALS/FTD and is one of the most promising therapeutic gene targets now known for sporadic ALS and FTD. The mouse models provided herein, in some embodiments, may be used to develop an antisense ASO-based therapy targeted to restore stathmin-2 levels by blocking the aberrant splicing of stathmin-2 pre-mRNA. Utilizing an approach reminiscent of the ASO-mediated alteration in splicing of the SMN2 pre-mRNA in SMA, for example, these mouse models may be used to test the therapeutic potential of stathmin-2 ASOs in sporadic and familial ALS/FTD.

Candidate therapeutic molecules of the present disclosure also include putative or known small-molecule modulators (e.g., inhibitors, drugs) of stathmin-2 pre-mRNA or mRNA splicing.

Assaying or assessing modifications (changes) in a mouse phenotype are known as are the various phenotypes associated with neurodegenerative disease models. The phenotype may be, for example, a behavioral phenotype or a pathological (e.g., neuropathological) phenotype (see, e.g., Kang J. et al. STAR Protoc. 2021-7-7; 2(3):100654). See also Janus C et al. Methods Mol Biol. 2010; 602:323-45; Choi J. et al. NMR Biomed. 2007; 20:26-237; van der Staay F. et al. Behav Brain Funct. 2009-2-25; 5:11.

In some embodiments, the phenotype is a motor deficit. Non-limiting examples of motor deficits include tremors, paralysis, abnormal gait, and hindlimb clasping. In some embodiments, the motor deficit is assayed by open field, grip strength, or rotarod analyses.

In some embodiments, the phenotype is a cognitive deficit (see, e.g., Holter S. et al. Curr Protoc Mouse Biol. 2015-12-2; 5(4):331-358).

Other phenotypes that may be assessed include motor neuron degeneration and/or neuromuscular denervation.

### Additional Embodiments

The following numbered embodiments are encompassed by the present disclosure:
1. A mouse comprising a nucleic acid encoding a truncated Stathmin-2 messenger ribonucleic acid (mRNA).
2. The mouse of embodiment 1, wherein the truncated Stathmin-2 mRNA lacks a polynucleotide sequence encoded by exons 2-5 of an endogenous mouse Stathmin-2 gene.
3. The mouse of embodiment 1 or 2, wherein the truncated Stathmin-2 mRNA comprises a region of a human Stathmin-2 mRNA.
4. The mouse of embodiment 3, wherein the region of the human Stathmin-2 mRNA is encoded by a human Stathmin-2 exon 2a polynucleotide sequence.
5. A mouse comprising a nucleic acid comprising a region of a human Stathmin-2 gene.
6. The mouse of embodiment 5, wherein the region comprises a human Stathmin-2 exon 2a polynucleotide sequence and a polyadenylation sequence.
7. A mouse comprising an endogenous Stathmin-2 gene locus, wherein the endogenous Stathmin-2 gene locus comprises a nucleic acid comprising a human Stathmin-2 exon 2a polynucleotide sequence and optionally a polyadenylation sequence.
8. The mouse of embodiment 4, 6, or 7, wherein the human Stathmin-2 exon 2a polynucleotide sequence has a length of 350-450 nucleotide base pairs.
9. The mouse of embodiment 8, wherein the human Stathmin-2 exon 2a polynucleotide sequence comprises the nucleotide sequence of SEQ ID NO: 6 or a sequence having at least 90% or at least 95% identity to the nucleotide sequence of SEQ ID NO: 6.
10. The mouse of any one of embodiments 4 or 6-9, wherein the human Stathmin-2 exon 2a polynucleotide sequence is in an intronic region of the endogenous Stathmin-2 gene locus.
11. The mouse of embodiment 10, wherein the human Stathmin-2 exon 2a polynucleotide sequence is between exon 1 and exon 2 of the endogenous Stathmin-2 gene locus.
12. The mouse of any one of embodiments 4 or 6-11, wherein the human Stathmin-2 exon 2a polynucleotide sequence comprises a Tar DNA-binding protein 43 (TDP-43) binding site.
13. The mouse of any one of embodiments 4 or 6-11, wherein the human Stathmin-2 exon 2a polynucleotide sequence comprises a modified TDP-43 binding site that does not bind to TDP-43.
14. A method comprising:
   administering to the mouse of any one of embodiments 1-13 a candidate therapeutic molecule; and
   assaying for a modified phenotype in the mouse.
15. The method of embodiment 14, wherein the candidate therapeutic molecule blocks aberrant splicing of Stmn2 pre-mRNA.
16. The method of embodiment 14 or 15, wherein the candidate therapeutic is an antisense oligonucleotide (ASO) that binds to the human exon2a polynucleotide sequence.
17. The method of embodiment 14 or 15, wherein the candidate therapeutic is a small molecule drug.
18. The method of any one of embodiments 14-17, wherein the phenotype is a behavioral phenotype or a pathological phenotype.
19. The method of any one of embodiments 14-18, wherein the phenotype is a motor deficit.
20. The method of embodiment 19, wherein the motor deficit is selected from tremors, paralysis, abnormal gait, or hindlimb clasping.
21. The method of embodiment 19 or 20, wherein the motor deficit is assayed by open field, grip strength, or rotarod analyses.
22. The method of any one of embodiments 14-18, wherein the phenotype is a cognitive deficit.
23. The method of any one of embodiments 14-18, wherein the phenotype is motor neuron degeneration and/or neuromuscular denervation.
24. A method of producing the mouse of any one of embodiments 1-13 comprising introducing the nucleic acid into the genome of the mouse.

### Examples

### Example 1. Generation of Knock-In Humanized Stmn2 Mice

In order to determine how stathmin-2 contributes to neuronal function and to develop therapeutic approaches based on restoration of stathmin-2 levels in ALS and FTD, the field needs more faithful animal models where processing of the stathmin-2 transcript is dependent on TDP-43 function, as has demonstrated to be true in human. In this Example, ALS/FTD mouse models were generated in which a portion of intron 1 of the stathmin-2 gene has been humanized. This approach can be used to evaluate the degree of TDP-43 dysfunction in different ALS/FTD models and establish whether abnormal stathmin-2 mRNA processing contributes to neurodegeneration.

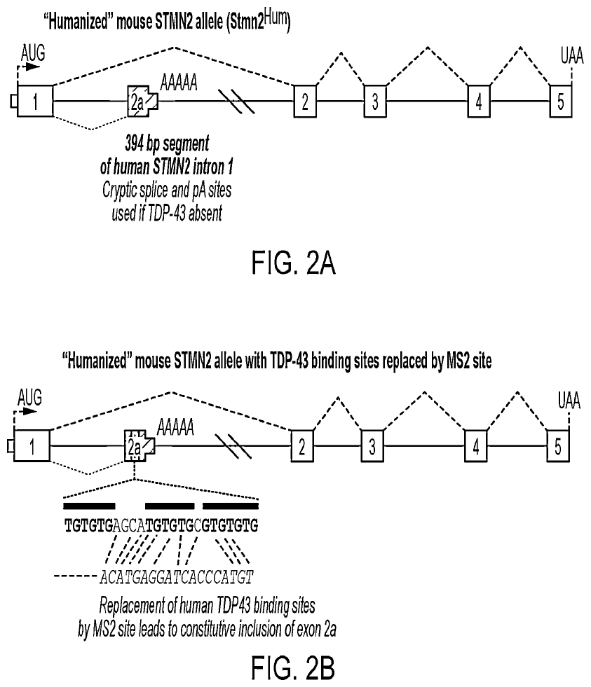

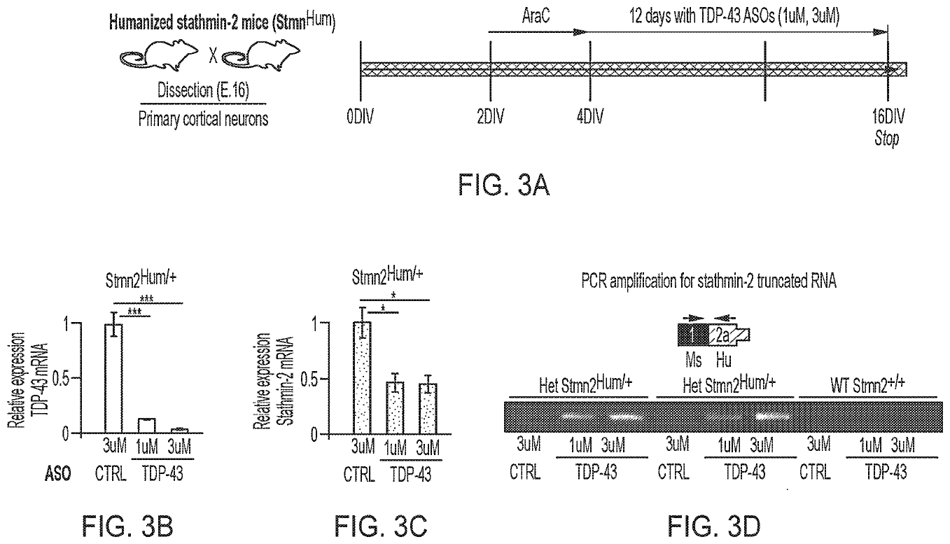

As discussed above, the mouse Stmn2 gene does not contain an exact homolog to human exon 2a. It does, however, contain a pseudo exon 2a region within intron 1 of the Stmn2 gene that contains a putative (but unused) splice sequence, but it lacks both TDP-43 binding sequences and a polyadenylation signal. In order to generate a rodent model recapitulating the TDP-43-dependent cryptic splicing and premature polyadenylation-driven loss of stathmin-2, CRISPR/Cas9 genome-editing was used to replace a 479 nucleotide (nt) region of mouse intron 1 containing the pseudo exon 2a with a 394 nt human sequence that contains the 227 bases of human exon 2a with 75 and 92 flanking bases (FIG. 2A). Correct genome editing was verified by short and long-range PCR followed by sequencing. It was then determined that both heterozygously and homozygously humanized Stmn2 mice (herein referred as Stmn2^{Hum/+} and Stmn2^{Hum/Hum}) are viable and fertile. Using cultured primary cortical neurons from these Stmn2^{Hum/+} mice (FIG. 3A), the processing of the humanized Stmn2 pre-mRNA was validated as dependent upon TDP-43. Indeed, ASO-mediated reduction of TDP-43 in these humanized primary neurons (FIG. 3B) elicited reduction in full length stathmin-2 mRNA (FIG. 3C), with both cryptic splice and polyadenylation sites used to produce a truncated Stmn2 mRNA (in which mouse exon 1 was spliced into human exon 2a—FIG. 3D).

### Example 2. Validation of Cryptic Splicing/Polyadenylation of Stathmin-2 Pre-mRNA

While in vitro preliminary results strongly support that inserting the sequence of the human exon 2a in the endogenous mouse stathmin-2 gene efficiently recapitulates TDP-43 dependency of stathmin-2 pre-mRNA processing, this result will be validated in the adult nervous system using ICV injection in P1 Stmn2^{Hum/Hum} pups of AAV9 expressing TDP-43 shRNA or a control shRNA. Tissues will be harvested 4 weeks after injection to measure TDP-43 and full length stathmin-2 RNA and protein levels by immunoblot and quantitative RT-PCR. Production of truncated exon 2a-containing Stmn2 RNAs will be assessed in RNA extracted from cortex, hippocampus and cerebellum (n=6 per group). An alternative to shRNA-mediated reduction of TDP-43 will be to use ICV injection of ASOs targeting mouse TDP-43 mRNA in adult mice, a method previously reported to be well tolerated and transiently reduce TDP-43 levels in the central nervous system.

### Example 3. Blocking of TDP-43 Binding to Stmn2 Induces Constitutional Misplicing and Reduction of Stmn2 in Mouse Cells with Humanized Exon 2a

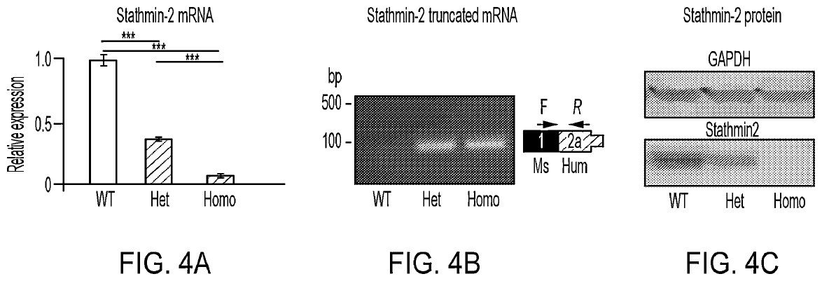

Humanization of the region encoding exon 2a in the mouse endogenous gene (FIG. 2A) induced cryptic splicing upon TDP-43 reduction (FIGS. 3A-3D). However, an essential validation beyond such cellular systems is to enable identification of the most efficient ASOs targeting stathmin-2 cryptic splicing in the true in vivo context of the mammalian nervous system. To achieve this, in vivo screening of drugs targeting stathmin-2 would be facilitated by a mouse model with constitutional stathmin-2 misplicing that does not require TDP-43 disruption. For this, testing was performed to determine whether mutating the TDP-43 binding sites led to stathmin-2 misprocessing in mouse N2a neuronal cells engineered to carry a humanized exon 2a. A CRISPR/Cas9 engineering approach was used to replace the 24 nucleotides containing the three GU-motifs bound by TDP-43 with a 19 nucleotide MS2 aptamer sequence known to adopt an RNA stem-loop structure which is bound with high affinity by the MS2 coat protein (MCP) (FIG. 2B). Mutating the TDP-43 binding sites in one or both alleles of the chimeric human/mouse endogenous stathmin-2 gene (FIG. 2B) led to chronic production of truncated stathmin-2 transcripts (FIG. 4B) and reduced levels of full length stathmin-2 mRNA (FIG. 4A) and protein (FIG. 4C). Thus, a novel mouse model for efficient in vivo testing of drugs targeting stathmin-2 misprocessing has been generated.

Mice have been generated with a humanized stathmin-2 allele in which a 24-base segment of exon 2a containing the three TDP-43 binding sites has been replaced by a 19 base MS2 binding site (FIG. 2B). Two founders were identified, and lines are now being expanded. With these, the viability of heterozygous and homozygous mice (Stmn2^{Hum-ΔGU/+} and Stmn2^{Hum-ΔGU/Hum-ΔGU}), will be determined, then RNA and protein extracted from different brain regions of 1 month-old animals (n=5 per group, both sexes) to determine whether, as observed in N2a cells (FIGS. 4A-4C), stathmin-2 is misprocessed independently of TDP-43 function.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A mouse comprising a humanized stathmin-2 (Stmn2) gene comprising an exogenous polynucleotide sequence, wherein the exogenous polynucleotide sequence comprises human STMN2 exon 2a sequence.

2. The mouse of claim 1, wherein the human STMN2 exon 2a sequence has a length of about 200 to about 300 nucleotides, optionally a length of about 220 to about 225.

3. The mouse of claim 1 or 2, wherein the exogenous polynucleotide sequence comprises human genomic DNA flanking the human STMN2 exon 2a sequence.

4. The mouse of any one of the preceding claims, wherein the human STMN2 exon 2a sequence comprises a human MS2 stem loop sequence.

5. The mouse of claim 4, wherein the human MS2 stem loop sequence comprises the sequence of 5′-ACATGAGGATCACCCATGT-3′ (SEQ ID NO: 4).

6. The mouse of claim 5, wherein the human MS2 stem loop sequence replaces TDP-43 binding sequence.

7. The mouse of claim 6, wherein the TDP-43 binding sequence comprises the sequence of 5′-TGTGTGAGCATGTGTGCGTGTGTG-3′ (SEQ ID NO: 5).

8. The mouse of any one of the preceding claims, wherein the human STMN2 exon 2a sequence is in intron 1 of the mouse Stmn2 gene.

9. The mouse of any one of the preceding claims, wherein The mouse has a C57BL/6 genetic background.

10. The mouse of claim 9, wherein the genotype of the mouse is C57BL/6J-Stmn2^{em8(STMN} [symbol] ^{)Lutzy}/Mmjax.

11. The mouse of any one of the preceding claims, wherein the mouse exhibits one or more symptoms of a Tar DNA-binding protein 43 (TDP-43) proteinopathy, optionally wherein the TDP-43 proteinopathy is selected from amyotrophic lateral sclerosis (ALS) and frontotemporal degeneration (FTD).

12. A method comprising administering a candidate therapeutic agent to the mouse of any one of the preceding claims, and assaying the mouse for a modified phenotype.

13. The method of claim 12, wherein the candidate therapeutic agent blocks aberrant/cryptic splicing of Stmn2 pre-mRNA.

14. The method of claim 12 or 13, wherein the candidate therapeutic is an antisense oligonucleotide (ASO) that binds to the human exon2a polynucleotide sequence.

15. The method of claim 12 or 13, wherein the candidate therapeutic is a small molecule drug.

16. The method of any one of claims 12-15, wherein the phenotype is selected from a behavioral phenotype, a pathological phenotype, a cognitive deficit, a motor deficit, motor neuron degeneration, neuromuscular denervation.

17. The method of claim 16, wherein the phenotype is a motor deficit.

18. The method of claim 17, wherein the motor deficit is selected from tremors, paralysis, abnormal gait, or hindlimb clasping.

19. The method of claim 17 or 18, wherein the motor deficit is assayed by open field, grip strength, or rotarod analyses.

20. The method of any one of claims 12-15, wherein the phenotype is a cognitive deficit.

21. A method of producing the mouse of any one of claims 1-11, the method comprising introducing the exogenous polynucleotide sequence into an intron of the endogenous Stmn2 gene of the mouse, optionally using a gene editing technique.
